(19) United States
(12) Patent Application Publication
(10) Pub. No.: US 2006/0161251 A1
(43) Pub. Date: Jul. 20, 2006

(54) Cuffs for medical applications

(76) Inventor: David Peter Shaw, Christchurch (NZ)

Correspondence Address: WELLS ST. JOHN P.S., 601 W. FIRST AVENUE, SUITE 1300, SPOKANE, WA, 99201, US

(21) Appl. No.: 10/561,886

(22) PCT Filed: Jun. 22, 2004
(86) PCT No.: PCT/NZ04/00130

§ 371 Date: Dec. 20, 2005

(30) Foreign Application Priority Data
Jun. 30, 2003 (NZ) 526775

(51) Int. Cl. A61F 2/24

(52) U.S. Cl. 623/2.41; 623/2.36; 606/151

(57) ABSTRACT

A cuff for medical applications, which may be sewn, glued or frictionally fitted in place; the cuff is made of a flexible openwork structure of a medically acceptable metal such as titanium or a titanium alloy; the flexible openwork structure may be e.g. manufactured from wire using a knitting, weaving or chainmail type structure.

### TECHNICAL FIELD

The present invention relates to cuffs for medical applications, i.e. to cuffs which surround components which have to be positioned in the body. Some cuffs are sewn in place, others are simply positioned around other components.

### BACKGROUND ART

At present, cuffs usually are made of fabric, generally a very tightly woven Dacron (trademark) material which is both dense and flexible. Also, Dacron can be readily penetrated by a sewing needle if the cuffs are sewn in.

However, fabric cuffs present a huge surface area and hence a large number of sites in which bacteria can hide from the body's defences; this greatly increases the risk of infection, and makes any infection which does occur, very difficult to treat. A further drawback of fabric cuffs is that the body regards the fabric as a “foreign body”, so that if the cuff is infected, it is almost impossible to sterilise with antibiotics or for the body's defences to phagocytose on the surface of the fibre.

Thus, if a fabric cuff becomes infected it generally is necessary to replace the cuff, and often to replace both the cuff and the component being held in place by the cuff. This involves an additional surgical procedure on the patient who may already be seriously ill, which obviously is undesirable.

### DISCLOSURE OF INVENTION

It is an object of the present invention to provide a cuff for medical use which overcomes the above described drawbacks.

The present invention provides a cuff for medical use wherein the cuff is made of a flexible openwork structure of a medically acceptable metal.

As used herein, the term “medically acceptable” means a metal which is non-toxic to the body and preferably which is inert in the body, i.e. does not provoke a “foreign body” reaction when implanted in the body. It is envisaged that the cuff of the present invention suitably would be made from titanium or medically approved titanium alloys (for example the nickel/titanium Nitenol (trademark) alloys), but other medically acceptable metals could be used.

The flexible openwork structure may be made from a thin plate cut with multiple perforations to give a flexible structure with a large number of apertures. It is important that the edges of the apertures are not sharp, so that they do not cut the sewing thread; laser cutting of the apertures may be used to give acceptably smooth edged apertures.

Alternatively, the flexible openwork structure can be made from wire, e.g. by using a knitting type of process or by a weaving process, or by manufacturing chain mail, (i.e. a series of separate interlocked rings of wire), or by using a ‘steel wool’ type of structure.

The finished openwork structure must be able to flex without permanently bending and (in the case of a sewn cuff) must provide a large number of apertures through which a sewing needle can be inserted.

### BRIEF DESCRIPTION OF DRAWINGS

By way of example only, preferred embodiments of the present invention are described in detail with reference to the accompanying drawings in which:

FIGS. 1 and 2 show perspective views of the upper and lower surfaces respectively of a replacement heart valve fitted with a sewn-in cuff in accordance with the present invention;

FIG. 3 shows a sketch side view of a cuff in accordance with the present invention used in connection with a line; and

FIG. 4a-e show sections of knitted wire, woven wire, chainmail, steel wood, and perforated plate, respectively.

### BEST MODE FOR CARRYING OUT THE INVENTION

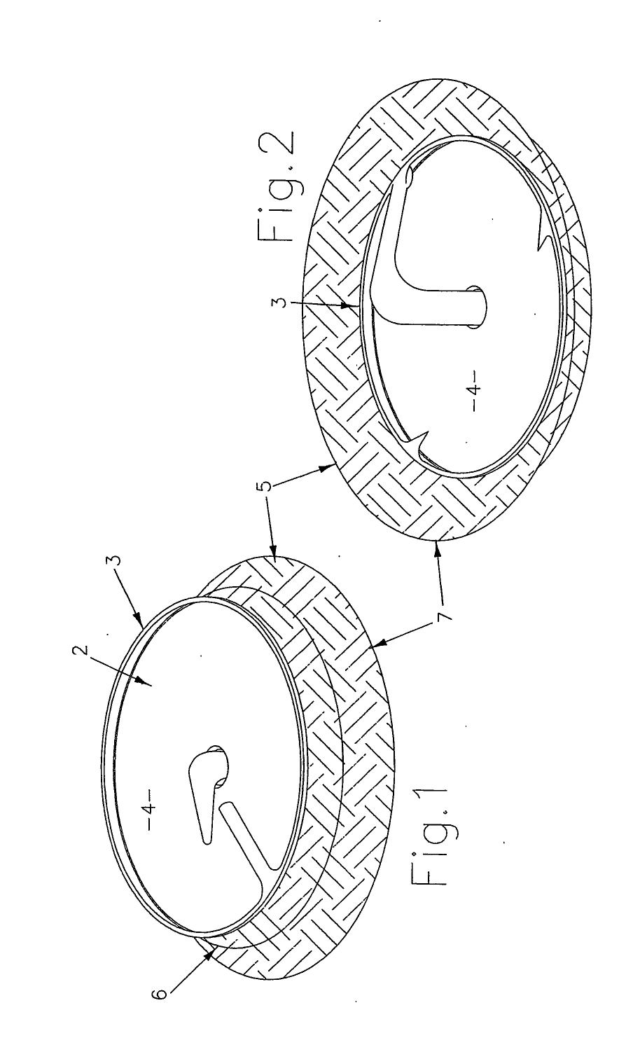

Referring to FIGS. 1 and 2, a widely used design of replacement heart valve 2 consists of a titanium rim 3 which provides support for the valve flap 4. The heart valve itself is a ‘Medtronic Hall’ mechanical valve of known design and will not be described in detail.

The rim 3 supports a cuff 5 made of knitted titanium wire. The cuff 5 is the same basic shape as the Dacron cuff it replaces: an inner annulus 6 which fits tightly around the rim 3, holding the cuff 5 onto the valve 2, and an outer annulus 7, which is formed integrally with the inner annulus 6 but which is of larger diameter.

The cuff 5, because of its knitted construction, is flexible and provides a very large number of apertures through which a sewing needle can be inserted, to sew the cuff into the body. The cuff thus provides a secure but flexible seating for the valve. The cuff can be sewn into place as easily as the Dacron cuff it replaces, but is very much less prone to bacterial infection and, if it becomes infected, can be sterilised effectively with antibiotics, without resorting to surgery.

A further advantage is that titanium and titanium alloys not only are regarded as inert by the body, but promote good tissue growth. Thus, as the body heals around the inserted valve, tissue will readily grow over and into the cuff, reducing the incidence of paravalvular leaks. In addition, it is envisaged that the cuff of the present invention will provide superior endothelisation, reducing thrombenbolic rates and giving reduced pannus formation.

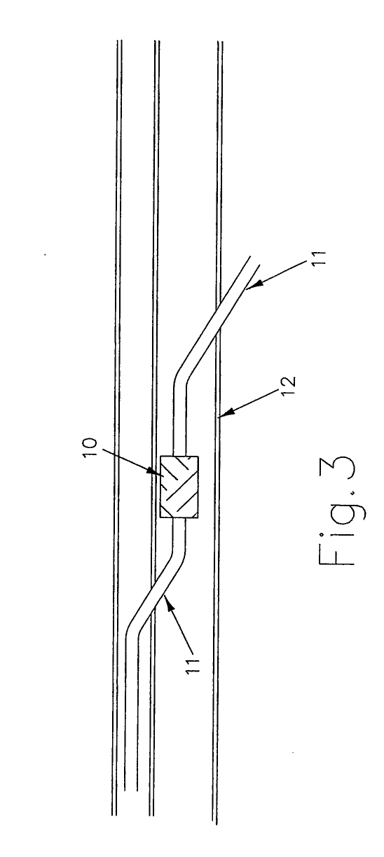

Referring to FIG. 3, a cuff 10 in accordance with the present invention is shown in use in combination with a line 11. The line 11 may be e.g. an intrathoracic line or an intravenous line or an intraperitoneal line. The line 11 is inserted through the skin 12 in known manner and the cuff 10 is located just below the skin, and encircles the outer surface of the line, to prevent infection entering the body through the aperture which admits the line 11, and travelling down the outer surface of the line.

The cuff 10 consists of a cylinder formed from knitted titanium wire. The inner diameter of the cylinder is such that the cylinder can be press fitted over the line 11. The cuff 10 is secured in place by frictional contact with the exterior of the line or by adhesive.

It will be appreciated a cuff formed in accordance with the present invention may be used in any of a wide range of applications where at present fabric cuffs are used, and is not limited to the particular applications described in detail above. For example, cuffs in accordance with the present invention may be used as barrier cuffs in combination with peritoneal dialysis catheters, held in position by frictional contact. Further, the cuffs of the present invention may be used to form annuloplasty bands or rings (a band being an incomplete ring) which are sewn in place and used to tighten an annulus or support an annulus after valve repair.

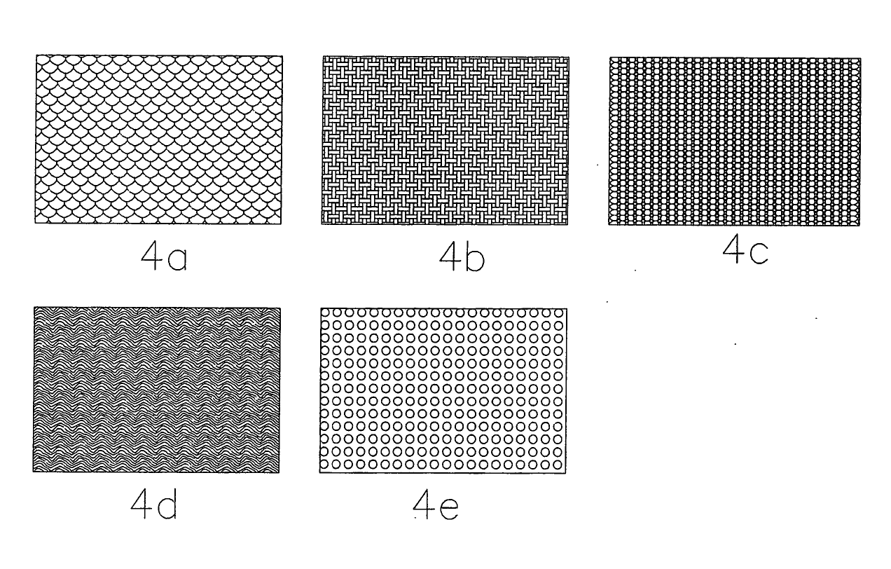

The above described cuffs 5 and 10, rather than being made of a knitted construction of the type shown in FIG. 4a may instead be made from a woven wire structure (FIG. 4b) or as chainmail, i.e. a series of interlocked rings of wire, as shown in FIG. 4c, or a random “steelwool” type of structure as shown in FIG. 4d. A further possibility is to make the cuffs of a perforated plate as shown in FIG. 4e: this is a thin plate cut with multiple perforations, all the perforations having rounded edges so that they do not cut any sewing sutures. The plate must be thin enough that the resulting perforated plate is flexible.

1: A cuff for medical use wherein the cuff is made of a flexible openwork structure of a medically acceptable metal.

2: The cuff as claimed in claim 1 wherein the cuff is formed to provide one of the following: a cuff for a heart valve; a cuff for a line; a barrier cuff for a peritoneal dialysis catheter; an annuloplasty band; an annuloplasty ring.

3: A cuff for a mechanical heart valve, wherein the cuff is made of a flexible openwork structure of a medically acceptable wire and provides an inner annular rim dimensioned to fit around the perimeter of the heart valve and formed integrally with an outer annulus of larger diameter than said inner annular rim.

4: The cuff as claimed in claim 1, wherein the flexible openwork structure is selected from one of the following: knitted wire, woven wire, wire fabricated into a chainmail structure, wire formed into a random “steel wool” structure.

5: The cuff as claimed in claim 4, wherein the metal is selected from: titanium; a medically approved titanium alloy.

6: A method of promoting tissue ingrowth and endothelialisation and minimising the risk of foreign body infection following the insertion of a cuff in a living subject, said method comprising providing a cuff made of a flexible openwork structure of a medically acceptable metal.

7: A method of promoting tissue ingrowth and endothelialisation, minimising the risk of foreign body infection and minimising paravalvular leaks following the fitting of a prosthetic heart valve mounted upon a cuff, in a living subject, and said method comprising the provision of a prosthetic heart valve having a peripheral cuff made of a flexible openwork structure of a medically acceptable metal.

8: The method as claimed in claim 7, wherein the cuff provides an inner annular rim dimensioned to fit around the perimeter of the heart valve and formed integrally with an outer annulus of larger diameter than said inner annular rim.

9: The method as claimed in claim 6, wherein the flexible openwork structure is selected from one of the following: knitted wire, woven wire, wire fabricated into a chainmail structure, wire formed into a random “steel wool” structure.

10: The method as claimed in claim 9, wherein the metal is selected from: titanium; a medically approved titanium alloy.

11: The cuff as claimed in claim 2, wherein the flexible openwork structure is selected from one of the following: knitted wire, woven wire, wire fabricated into a chainmail structure, wire formed into a random “steel wool” structure.

12: The cuff as claimed in claim 3, wherein the flexible openwork structure is selected from one of the following: knitted wire, woven wire, wire fabricated into a chainmail structure, wire formed into a random “steel wool” structure.

13: The cuff as claimed in claim 11, wherein the metal is selected from: titanium; a medically approved titanium alloy.

14: The cuff as claimed in claim 12, wherein the metal is selected from: titanium; a medically approved titanium alloy.

15: The method as claimed in claim 7, wherein the flexible openwork structure is selected from one of the following: knitted wire, woven wire, wire fabricated into a chainmail structure, wire formed into a random “steel wool” structure.

16: The method as claimed in claim 8, wherein the flexible openwork structure is selected from one of the following: knitted wire, woven wire, wire fabricated into a chainmail structure, wire formed into a random “steel wool” structure.

17: The method as claimed in claim 15, wherein the metal is selected from: titanium; a medically approved titanium alloy.

18: The method as claimed in claim 16, wherein the metal is selected from: titanium; a medically approved titanium alloy.
